(19) United States
(12) Patent Application Publication
(10) Pub. No.: US 2022/0270511 A1
(43) Pub. Date: Aug. 25, 2022

(54) NEUROSCIENCE CONTROLLED VISUAL BODY MOVEMENT TRAINING

(71) Applicants: Andrew John Blaylock, Minneapolis, MN (US); Jeffrey Thielen, Lino Lakes, MN (US)

(72) Inventors: Andrew John Blaylock, Minneapolis, MN (US); Jeffrey Thielen, Lino Lakes, MN (US)

(21) Appl. No.: 17/675,675

(22) Filed: Feb. 18, 2022

Related U.S. Application Data
(60) Provisional application No. 63/151,330, filed on Feb. 19, 2021.

(51) Int. Cl. G09B 19/00; G09B 5/06; A61B 5/11

(52) U.S. Cl. CPC G09B 19/0038; G09B 5/065; A61B 5/1114; A61B 5/1118; A61B 2503/10

(57) ABSTRACT

Embodiments of a system and method for retrieving a skill training data file, outputting the skill training data file for display on a user interface, and displaying the skill training data file are generally described herein. The skill training data file may include a set of video portions displayed in a sequence including a first passive video portion. The skill training data file may include a second video portion including visual or audible instructions directing a viewer to imagine body sensations corresponding to images shown in the second video portion. In some examples, the body sensations may be representative of muscle movements used to perform the skill.

### CLAIM OF PRIORITY

This application claims the benefit of priority to U.S. Provisional Application No. 63/151,330 filed Feb. 19, 2021, titled “NEUROSCIENCE CONTROLLED VISUAL BODY MOVEMENT TRAINING,” which is hereby incorporated herein by reference in its entirety.

### BACKGROUND

Human brains are amazingly powerful associative learning machines. If two phenomena hit a person's sensory systems consistently together in time, the brain may create an associative memory to link those two phenomena. To the degree that those two phenomena themselves contain parsimonious information (internal structure), the brain may find correlated patterns within said phenomena and may encode deep structural relationships between those phenomena. This apparently happens automatically and effortlessly.

When a human creates movement, two data streams are available to them. One is the output motor patterns (which themselves generate predictions about the sensory information expected to result from those motor actions) and the other is returning sensation. A lot of learning is achieved by comparing these two streams.

However, consider how salient returning sensory information during a motion is compared to a visual of that same motion when it comes to the tiny details about what happened. The visual seems to provide more value when it comes to analyzing the motion in detail.

The implication of this is implemented in weight rooms all around the world. Mirrors are installed so that people can execute, feel, and see their motion all at the same time. Mirrors work well, but offer minimal flexibility in terms of angle of view and other features.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a video playback application in accordance with some examples.

FIG. 2 illustrates video sequences in accordance with some examples.

FIG. 3 illustrates a block diagram of a data file structure in accordance with some examples.

FIG. 4 illustrates a flowchart showing a technique for skill development video processing in accordance with some examples.

FIG. 5 illustrates a flowchart showing a technique for skill training data file retrieval and display in accordance with some examples.

FIG. 6 illustrates a block diagram of an example of a machine upon which any one or more of the processes discussed herein may perform in accordance with some embodiments.

### DETAILED DESCRIPTION

When we learn skills, they can be of a few different types. One type of skill is a cognitive skill and this is of a type where the brain is trained so as to take in a given input (often in the form of a question) and an output comes to mind (often in the form of an answer to the question). In this case, the process may or may not produce a physically measurable change in the world, but the holder of the skill does feel a change of state of mind after using the skill in that the output is “brought to mind”.

Another type of skill is a procedural skill where the holder of the skill has a memory of a series of steps needed to accomplish some task. At first, they have to consciously step through the series of steps in order to execute the skill. Over time, executing a procedural skill is less and less dependent on conscious effort and the series of steps becomes more and more automatic. At that point, the skill can seem to be more of a unified whole and the procedural skill begins to look similar to a cognitive skill as described above.

However, some procedural skills produce physical effects in the world through the use of the holder of the skill's muscles. These are procedural movement skills and they are, like other procedural skills, susceptible to becoming more and more an automatic unified whole through training.

One way of measuring the physical effects of a procedural movement skill is with a video camera. Taking a video of a person executing a movement captures the details of that motion for further processing into useful quantitative data as desired. And, in the present disclosure, we intend to use a specific type of further processing in the mind of the user of said disclosure to train that user toward improvement at the skill in question.

This method of learning by watching is called Observational Learning or “OL”. It can be done by watching a human expert in-person or by watching a video of an expert. In-person watching has advantages, but video does as well. Advantages for video include slow motion playback and repetitions on demand.

Human beings have learned via OL, probably for as long as there have been Homo Sapiens present on earth. Possibly, the method goes back even further than that. Most every sighted person can remember a moment in their lives where they used observation and mimicry to acquire a skill they had previously lacked or to refine a skill that needed improvement.

In addition, less intuitively, people have been using the power of Mental Imagery or “MI” to acquire or refine their movement skills. MI is the use of the imagination to simulate some experienced pattern using one or more of the sensory modalities. For most people, the first sense that may come to mind to use in a context of learning through MI is vision. This plays right in with the lesson from OL that the visual sense can significantly aid in the learning process. We call this Visual Mental Imagery or V-MI.

Other senses can play a role as well, including ones within the general category of interoception which is constituted by the interior body senses. When these interoception senses are related to body movement, they constitute one's Kinesthetic sense and include proprioception and stress-levels at the joints among other examples. Just as with V-MI, one can simulate the experience of Kinesthetic sense using their imagination. Doing this is called Kinesthetic Mental Imagery or K-MI.

Let us consider a common phenomenon one experiences while watching international-level performers in certain sports. Here we are pointing to the experience of watching the Olympics or world championships and seeing athletes with their eyes closed doing miniature versions of the movements involved in their sport immediately before they are to start the competition. These are experts and they have tremendous experience with what happens during the course of an elite-level competition. They also have had a huge amount of coaching to support that experience. This means they are also experts at what to imagine during MI preparation for a competition.

What about the rest of us? We do not have expertise at that level with respect to the sports we may want to learn. What should we imagine? At what speed? From what viewing angle? Should we imagine technique, or decision making in gameplay?

Let us focus on that last question. The answer is that it depends on what you want to learn. Are you trying to improve technique? Then you should focus on technique. But, then, what constitutes good technique? This one is easier because we have already talked above about the idea that OL may involve observing someone that is particularly good at the technique in question. If you capture video of an expert, that may be a great basis for your MI.

And that leads to “guided MI”. In this case, real displayed imagery is used to assist the user in controlling the quality of their M1. This solves the problem of what to imagine, at least for V-MI where the guiding phase may be prior to an imagining phase. However, it can also assist with K-MI. In K-MI, the video guidance and the work done by the imagination may happen concurrently. Using video as a touchstone that a viewer can refer to in cognitively controlling their MI seems to be particularly powerful. A shorthand for this is OL+MI and when the MI is specifically kinesthetic, OL+K-MI.

Cognitive control is a particularly challenging aspect of any form of training that is largely embodied in the realm of one's mind. It is understood that this sort of cognitive control is largely the challenge behind and the long-term aim, for some, of meditation. However, one who wishes to benefit from MI in their physical skill acquisition likely may prefer a shortcut as opposed to spending years improving their meditation skill in order to gain the cognitive control necessary to then do MI training related to a technique, and only then, to see the targeted benefit.

So, we see two benefits that a user can enjoy by using an engineered OL+MI system. First, the system can provide some of the best achievable imagery for a non-expert to use in acquiring a skill. Second, the guiding imagery of the OL part of OL+MI can also ease the burden on cognitive control that may otherwise be an obstacle to improvement.

The present disclosure targets this dual benefit to a novice or moderately-skilled user so as to give them the benefit of MI that an expert, not only can, but visibly does enjoy as part of their attempt to optimize their training regimen.

In this mode, the user, in time with the action they are observing on the screen, imagines body sensations that they themselves may feel if they were performing the action they are observing. However, given the limits of the human attentional system (it is apparently not capable of paying full attention to all parts of the body and all sensations inbound from those body parts simultaneously), it pays to focus on specific kinematic events related to the technique to be learned as a target for their imagined sensations. This means not only focusing in on some small area of the body such as the wrist, the hand, or the left leg, but also focusing on only some specific part of the sensory experience from that body part, such as weight transfer, muscle strain, or spatial position (through proprioception).

Note that aspects of this sensation can be exaggerated in this modality, and, in particular, in terms of its duration. In order to comfortably focus on the imagination task, it helps to play the guiding visual imagery in slow motion. This means sensations that may be fleeting in an actual execution of the technique will sometimes persist for a longer duration in OL+K-MI. Yet, this need not always be the case as full speed display may be used as well.

Which sensations and body parts are to be focused on? The answer depends on the technique and more importantly depends on the critical kinematic events that, when done correctly, constitute expert executions of the technique. By keying the user on important sensations related to successful executions of these critical kinematic events, the user can be placed on a more direct path to expertise than can be expected under normal circumstances.

Preparation

A challenge of this OL+K-MI method is to avoid overwhelming the user. If the user is simultaneously learning the details of the motion in question, guessing as to what sensations they may experience under the circumstances prescribed by the exercise, and trying to focus on simulating those sensations such that they have an imagined experience of them, they are likely to struggle to sustain that experience. Therefore, some preparation in advance may ease the cognitive burden during OL+K-MI and enhance both its effectiveness and user satisfaction with the experience.

One piece of preparation which can be assumed to be in place for the user is experience either with the technique in question or of moving their body in somewhat similar ways. This serves as a reference for the user with respect to the sensations they may experience during technique execution. A designer of an OL+K-MI experience may address the possibility that the user does not have this sort of prior experience by advising the user to give the technique a few attempts. However, another approach is to simply assume that anyone interested in an OL+K-MI training exercise either has an ongoing program where they are working on the technique in question or are to be subsequently starting one. This assumption is apparently true in most cases.

Additional preparation can be delivered as part of the learning experience which includes OL+K-MI and comes in the form of simply starting with some OL about the technique in question. By repeatedly viewing, from a variety of camera angles and framings, with different playback speeds, live or animated representations of the human figure executing the technique, with or without the addition of graphics to highlight key kinematic events, a variety of supporting music, verbal cues, and sound effects to reinforce key kinematic events and to help hold the attention, the user internalizes the details of the technique. This allows them to place their conscious focus on generating the K-MI that they have been directed to focus on during their OL+K-MI exercise and thus make “experiencing” the sensations inherent to quality technique execution their primary aim during OL+K-MI.

Further reinforcement and internalization of the details of the technique may come from the application of an OL+V-MI exercise in advance of the OL+K-MI exercise. In the OL+V-MI case, it is the visual imagery that the user has been watching during OL that is the focus of the imagination. By forcing the user to generate the visual imagery they have just watched, it is more deeply engrained into their memory. In an OL+V-MI example, the user iterates between watching a certain visual representation of an expert-level version of a technique on a video and imagining that same visual representation without any subsequent input from the eyes.

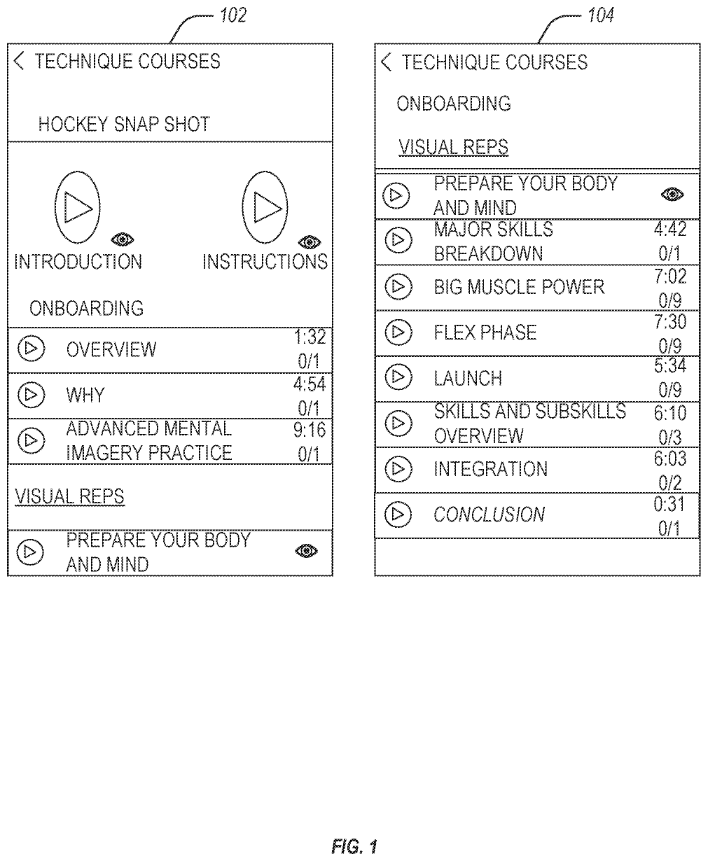

FIG. 1 illustrates a video playback application in accordance with some examples. FIG. 1 shows two instances of different user interfaces (UI) of the video playback application or two different video playback applications, illustrated as UI 102 and UI 104.

The UI 102 includes onboarding programming such as an overview section, and details of other aspects of video- and neuroscience-based skill learning. The UI 104 includes skill sections selectable within a visual reps category for skill learning. Segments within the skill categories on UI 104 may include observational learning or mental imagery videos.

Details and Variations

After any preparatory phase that may be used prior to the OL+K-MI exercise and then in an example, immediately before actually doing it, the user may be directed on what they are to be focusing on with their imagination. This may come in the form of verbal explanation as part of a video's audio track or visual instructions on a screen. It may be assisted by on-screen graphics that highlight the area of the body and the portion of the time-series of the technique which is the focus of the K-MI portion of the exercise.

In an example, in order to better hold the attention of the user, the system may vary the “treatment” (e.g., visual effects or text) of the on-screen visuals while repeating the same movement skill content over more than one on-screen “repetition” of the technique. Treatment options include the camera angles, framings, playback speeds, live or animated representations of the human figure executing the technique, use of graphics to highlight key areas, and use of music, verbal cues, sound effects, or the like.

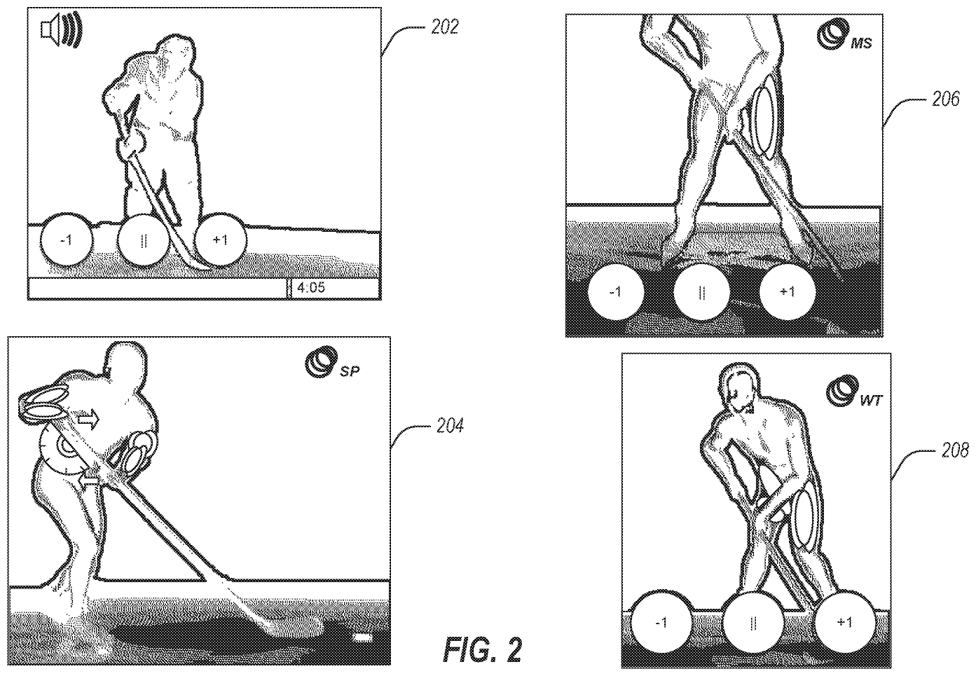

FIG. 2 illustrates video sequences in accordance with some examples. FIG. 2 includes four example video sequence screenshots 202, 204, 206, and 208. These screenshots are shown as examples, and are not necessarily used together or in any particular sequence. Example video sequence screenshot 202 illustrates a video portion, such as a passive video portion corresponding to a spatial position body sensation. Example video sequence screenshot 204 illustrates an active video of the spatial body sensation shown in the passive video of screenshot 202. Example video sequence screenshot 206 illustrates an active video for a muscle strain body sensation. Example video sequence screenshot 208 illustrates an active video for a weight transfer body sensation. A passive video portion may be called an observational video portion (e.g., without recommendation, requirement, or suggestion of an active movement or particular thought pattern).

In an example, a practitioner may develop a curriculum designed to teach a technique or set of techniques. They may partition this curriculum into courses which may be further partitioned into lessons. These lessons may be embodied in the form of individual video files played on a mobile, laptop, or other convenient display device common in modem homes.

A course, including at least one video (e.g., corresponding to screenshots 202, 204, 206, or 208), that implements an OL+K-MI method may be designed with an intrinsic order. It may be sequenced in such a way to give the user a sufficient amount of OL exposure before optionally also implementing a series of OL+V-MI exercises and then implementing OL+K-MI exercises.

One such sequence may focus the user on a critical kinematic event related to the technique and more specifically related to weight transfer. Initially the system may display repeated examples of that portion of the technique where such examples exhibit variety in its treatment as described above. Some of the variety of treatment seen in this OL sequence may include specific on-screen graphics that emphasize the nature of the weight transfer (e.g., video corresponding to screenshot 208), potentially including animated “pressure circles” underneath the on-screen representation of the human performing the technique's feet that shrink when there is little weight on a given foot and grow when there is a lot of weight on that foot. Alternatively, there may be a glow on the legs that dims or brightens to indicate an increase or decrease of load on that leg. Further along in this sequence an optional phase may be embodied by display of similar imagery that focuses the user on some key kinematic event interspersed with time where there is simply a black screen (OL+V-MI). The user may be directed to use their imagination to “experience” the same imagery that was just displayed to them during this black screen time. Audio may be repeated during the displayed-imagery portion and the black-screen portion to aid the user in producing imagined imagery that moves with the same timing as the viewed imagery. This sequence may conclude with a final phase which entails some description of how they are to imagine the weight transfer sensation that they may feel if they were the one performing the on-screen action they are about to see while watching the action and then displaying weight-transfer-focused imagery again. This weight transfer imagery may include graphics that direct the user's attention to the body part in question. Sound cues may serve to direct the user's attention to the body part and the target kinematic experience (in this case weight transfer) as well. For example, verbal cues may be used. A musical sequence or sound effect may be associated with the kinematic event in question (in this case, weight transfer felt in the legs) during the preparatory OL portion of the sequence and then that same sound can be used to reinforce the focus on that kinematic event during OL+K-MI.

The sequence described above need not be contained all in one video, it may be continuous within a video or continuous if spanning videos, it may start at the beginning of a video or end at the end of a video. That sequence represents the OL+K-MI with preparation design if a course contains the described elements in the order described above. Note that the OL+V-MI portion of the sequence is optional.

Another type of sequence may follow the same or a similar pattern as described above, including an OL phase, an optional OL+V-MI phase, and then an OL+K-MI phase with one or more repetitions of imagery related to a technique displayed in each of those phases. In this example, sound cues and on-screen graphics may call attention to the experience of muscle strain (e.g., video corresponding to screenshot 206) of some part of the body that may be experienced in some technique. In an example, the bottom hand on the stick in a hockey shot is to be pushed into the shaft of the stick as it is partially wedged against the ice on one end and held back against this push with the top hand. For a high velocity shot, this bottom hand push must be at near full power for the shooter and they can feel this activity in their muscles. The on-screen visual pattern along with the displayed auditory pattern in all three phases may call attention to this. For example, attention may be directed with sound cues noting the feeling of this muscle activity, graphical highlights which change the coloration or the brightness in this area around the muscles in question, or with motion graphics on top of or surrounding that area. As with the weight transfer version, it is important to note that during the OL+K-MI phase, sound cues may specifically direct the user to focus on imagining the muscle strain sensations of the part of the body in question while watching the on-screen imagery.

Another type of sequence may follow the same or a similar pattern as described above, including an OL phase, an optional OL+V-MI phase, and then an OL+K-MI phase with one or more repetitions of imagery related to a technique displayed in each of those phases. In this example, sound cues and on-screen graphics may call attention to the spatial position of some part of the body that may be experienced in some techniques. (e.g., video corresponding to screenshots 202 or 204). In this example, screenshot 202 may be part of the OL video (and optionally the K-MI phase) and screenshot 204 may be part of the K-MI phase. In an example, the top hand on the stick in a hockey shot may lead the puck as well as the user's mid-section (may be closer to the target), be just higher than the hip, and be slightly out front of the body. The on-screen visual pattern along with the displayed auditory pattern in all three phases may attention to this. For example, attention may be directed with sound cues noting this positioning, graphical highlights which change the coloration or the brightness in this area, or with motion graphics on top of or surrounding the area. As with the other versions, during the OL+K-MI phase sound cues may specifically direct the user to focus on imagining the spatial position of the part of the body in question while watching the on-screen imagery.

In an example, any or all of these types of OL+K-MI may be used in a single course over different videos.

In another example, two of the three of these types may be used within a single course over different videos.

In another example, one of these types may be used in a course where the sequence of components involved in OL+K-MI with preparation spans multiple videos.

In another example, any or all of these types may be used in a single video lesson.

In another example, two of the three of these types may be used within a single video lesson.

In another example, one of these may be used within a single video lesson.

An on-screen icon (for example, in the upper right corner of the screenshots 204, 206, 208) can be used that reminds the user what sensation or what body part they are to focus on while imagining the body sensations related to a technique (e.g., “SP” in 204, corresponding to spatial position). Examples include an icon for weight transfer, muscle strain, spatial position, or the like. In another example, an on-screen visualization of a human figure with an area of the body highlighted may be displayed. In this example, the user can, at a glance, recall what aspect of the motion that they are watching and are to be emphasizing within their mental imagery.

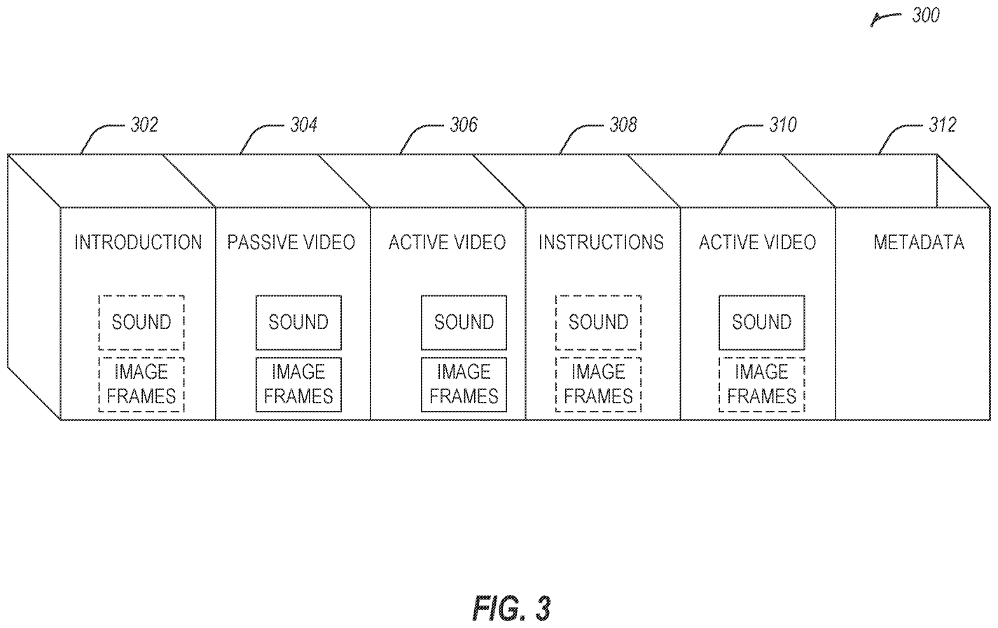

FIG. 3 illustrates a block diagram of a data file structure 300 in accordance with some examples. The data file structure 300 includes a plurality of data components. In an example, the data components are ordered (e.g., as shown). In another example, a data component maybe selected in any order to be played at a video playback device (e.g., the data file structure 300 is not necessarily ordered for playback).

The data components may include images, sound, both, or other data. For example, an introduction data component 302 may include only sound or only image frames, or both. A metadata component 312 may include other data, such as repetition information, menus, signaling, buffering information, control signaling, playback information, image and sound sync information, etc.

Playback components may include a passive video and sound component 304, including both sound and image frames, an active video and sound component 306 including both sound and image frames, and an active component 310 including only sound. In some examples, all image frames of the playback components 304, 306, and 310 may be the same. In other examples, each component may have unique images or share some images but not all. In some examples, sound for all playback components 304, 306, and 310 may be the same. In other examples, the sound may be modified for some components. In other examples, the sound may be unrelated among playback components.

An instructions component 308 may include image or sound information for display, such as instructions for a user related to one or both of the active video components 306 or 310. The instructions may include image or sound information to be overlaid or played with one or more other components, or may be played separately. The audio may act as a signal or as guidance for timing. The audio may be paired with or replaced by visuals that act as guidance. The guidance by audio or visuals may include identifying a body part for use during a particular portion of playback, or portion of the technique the user should be focused on with their imagination. The instructions may include this guidance or this type of guidance.

In an example, image frames and sounds for a playback component (e.g., passive video 304 or active video 306) may be tied together in the data file structure 300. For example, they may be stored together as a video file and pre-synced.

The image frames may include a single image, a series of images, or frames from a video file. The sound may include spoken word, music, sound effect, or the like. The sounds may be used to provide an emphasis (e.g., a weight transfer sound when changing feet, or a rhythm or a verbal cue, etc.) when a playback component is played.

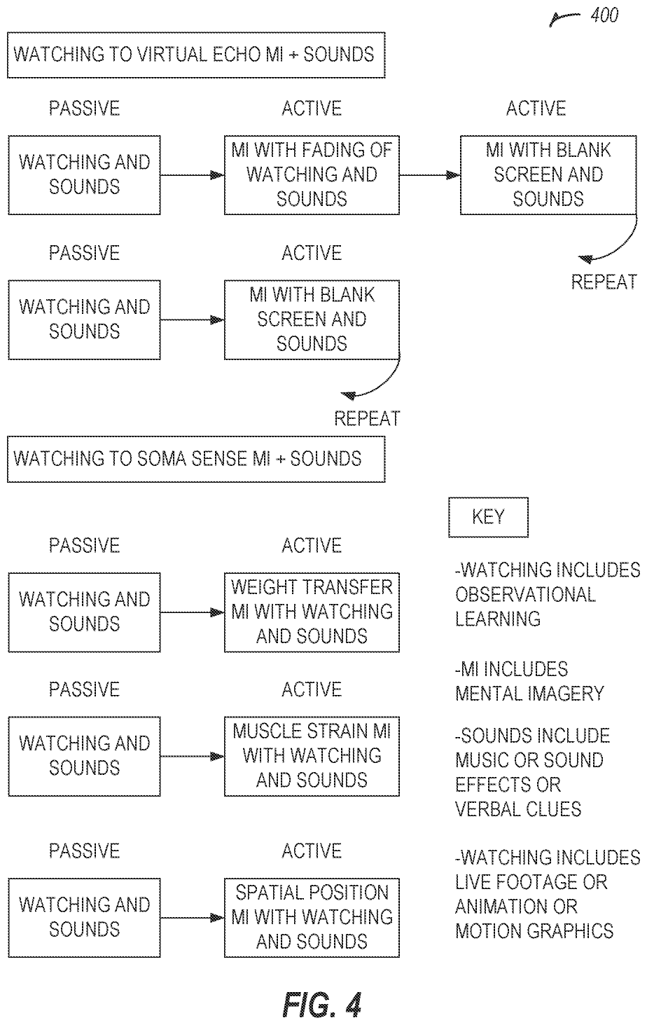

FIG. 4 illustrates a flowchart 400 showing a technique for skill development video processing in accordance with some examples. The flowchart 400 illustrates various options for video playback and instructions for developing a skill. For example, observational learning may precede mental imagery. FIG. 4 further shows passive and active operations in the flowchart 400. FIG. 4 illustrates five individual paths, which may be combined or interleaved in some examples. The paths may be repeated in some examples.

A first path includes passive video and sound, followed by active mental imagery with fading of video and sounds, followed by active mental imagery with a black or blank screen and sounds. A second path includes passive video and sound, followed by active mental imagery with a black or blank screen and sounds.

A third path includes passive video and sound, followed by active mental imagery including mental imagery of weight transfer body sensations with video and sounds. A fourth path includes passive video and sound, followed by active mental imagery including mental imagery of muscle strain body sensations with video and sounds. A fifth path includes passive video and sound, followed by active mental imagery including mental imagery of spatial position body sensations with video and sounds.

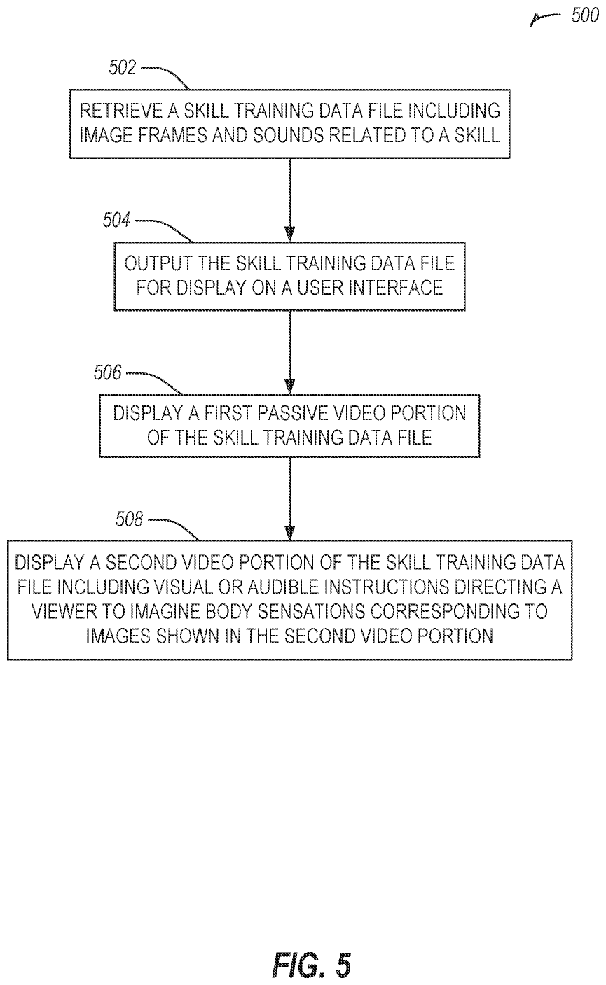

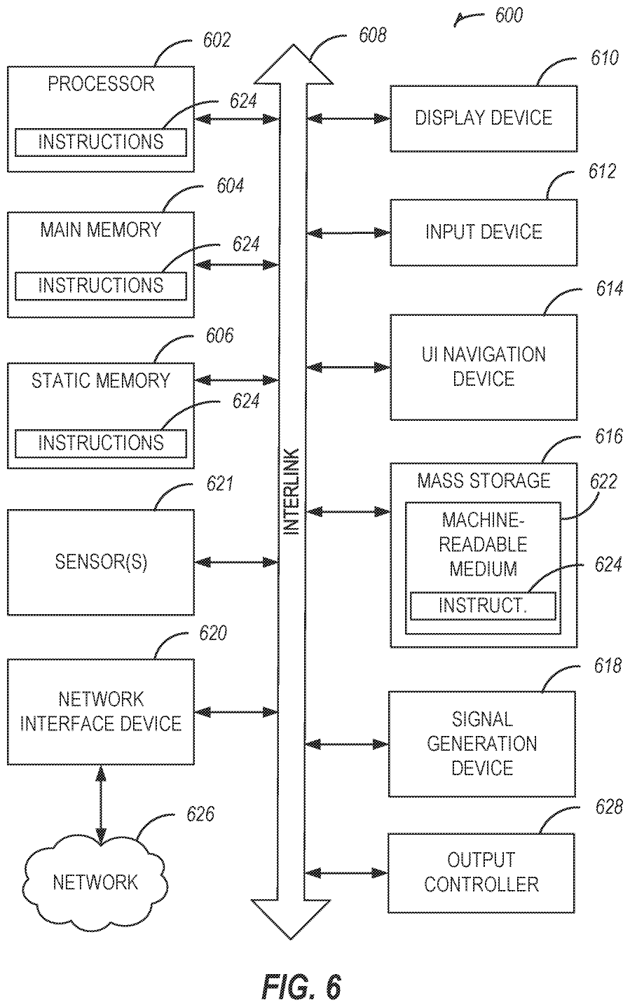

FIG. 5 illustrates a flowchart showing a technique 500 for skill training data file retrieval and display in accordance with some examples. The technique 500 may be performed using processing circuitry, such as a processor or processors of a device, such as a computer, a laptop, a mobile device or the like (e.g., as discussed in further detail below with respect to FIG. 6).

The technique 500 includes an operation 502 to retrieve a skill training data file including image frames and sounds related to a skill. The image frames may include different images in each video (e.g., different angles), which may be related to a specific technique and about a specific portion of the body and the time period of the technique. The image frames may include a set of images. In an example, a video may include a set of images. The set of images may be modified in minor ways, but still be considered the same video, in some examples. For example, a video or an image in the set of images may be modified by minor changes such as camera angle, field of view, coloring, speed, etc. Videos with these types of changes, but including the same underlying images, subject captured, sound, or concepts may be considered the same video in some examples disclosed herein.

Within the context of training a human movement skill, the factors that differentiate the inherent nature of a sensory stimulus may be categorized based on what that stimulus is about as opposed to its exact content. For example, an instructor may use the same word “go” to cue a student to start many different movements or even to switch between modes during a movement. In an example, a cornerback in football may backpedal as if playing coverage on a receiver, the instructor may then yell “go”, and the cornerback may, in response, switch to a mode of “driving hard on the ball” as if the quarterback has just made a throw. So, in different contexts, the word “go” may cause the student to do different things. On the other hand, many cues could all have that same effect for the student. For example, the instructor could say “drive” or “burst” instead of “go” in the example above and have the same effect. Audio cues may not be defined by their exact content, but instead by intent or the effects they may have on a student. In the context of audio cues and changes within a particular video that does not alter the underlying content or scene may include different types of audio that lead to the same effect.

This categorization of stimuli based on what it is about as opposed to the exact content may also apply for non-verbal audio cuing, visual cuing, and other forms of cuing by instructors for students. When those cues are delivered through an automated or pre-fabricated system such as a computerized device with a visual display and audio speakers, this is still true. In the case of video-based training, for example, focusing on the moving visual stimuli, the impact of a video sequence may be based on what human movement it shows, what portion of the movement in its time series it emphasizes, and what portion of the body that is moving it emphasizes. A video may emphasize an aspect of what the user would feel in terms of what sensory channel is to be focused on within that portion of the movement and portion of the body in considering how it would feel. It is these considerations which define what the video content is “about.” Video content that is alike along the lines of those variables but differs in camera angle, playback speed, coloration treatment, transparency level, zoom or pan, background audio, verbal cuing or other visual or audio design adjustment may be considered to be about the same video or same video content, and may be considered interchangeable in a training setting.

The technique 500 includes an operation 504 to output the skill training data file for display on a user interface. The skill training data file may include a plurality of video portions, such as a first passive video portion (e.g., video or audio only), a second video portion (e.g., video or audio plus additional instructions, which may be visual or audible, to a user to participate, such as by imaging the action displayed for the skill, imagining muscle movements, or the like).

The technique 500 includes an operation 506 to display a first passive video portion of the skill training data file. The technique 500 includes an operation 508 to display a second video portion of the skill training data file including visual or audible instructions directing a viewer to imagine body sensations corresponding to images shown in the second video portion. In an example, operations 506 and 508 may be performed sequentially in order with the first passive video portion played before the second video portion. The body sensations may be representative of muscle movements used to perform the skill, such as a weight transfer body sensation, a muscle strain body sensation, a spatial position body sensation, or the like. In an example, the instructions may be inherent in the visual display of the skill, displayed on a website before the video, be an active part can be displayed without the passive video, or the like.

The skill training data file may include an iteration of the first passive video portion, and the second video portion or the skill training data file may be replayed in some examples. The sounds may include music, and the music may be played during the second video portion or a third video portion at frames corresponding to when the music is played in the first passive video portion. The third video portion may be part of the skill training data file and include instructions to the viewer to imagine the first video portion without showing the first video portion (e.g., showing a blank screen, not showing anything on the screen, or showing only instructions on the screen).

In an example, portions of the skill training data file may be displayed in a particular order. In a first example, the order includes the first passive video portion, the third video portion (e.g., audio with a blank screen or no video), and the second video portion. In a second example, the order includes the first passive video portion, the second video portion, and the third video portion (e.g., audio with a blank screen or no video).

The second video portion may include the image frames and sound of the first passive video portion. The visual or audible instructions in this example, may be displayed or played before playing the image frames and sound, or concurrently with playing the image frames and sound.

Dynamic Templated Progression may be used to target a type of user behavior, which is not present in either physical practice or the consumption of traditional instructional videos. This behavior is the act of repeatedly engaging in observational learning (e.g., learning by watching) of content that is about the same subject matter as well as mental imagery tasks related to the visual system which are also about that subject matter, in a sustained way. This is an example of “visual reps”.

Generally, it is not difficult for users to sustain physical reps over time. There comes a threshold during any given practice session where a user may tend to feel that “enough is enough” and it becomes difficult to motivate oneself to execute more physical reps. However, there is often no resistance, or at least relatively less resistance, to going back later (the next day or within the next few days typically) and executing more physical reps.

This is typically not so with instructional videos (where some combination of verbal explanation and visual demonstration is used to create an understanding in the user's mind of how to execute expert-level technique for some movement skill). This is because most instructional videos do not aim for an effect related to “reps” targeted at the actual motor control areas of the brain. Instead, they are aimed at developing a “cognitive model”. To develop one's cognitive model is to learn, to some level of accuracy, an account of what happens when the technique is executed by a well-practiced expert. This cognitive learning can happen, mostly, over a single “repetition” of the video. What is learned can be used to guide future physical reps more accurately toward this expert version of the technique. A cognitive model does not, just by learning it, modify the ingrained movement pattern that the user already has or create a new movement pattern in users that do not already have one.

When the subject matter to be learned is a fact-based or narrative account of a phenomenon in the world, people do not feel a need for repetition of identical content. On the other hand, to fill holes in understanding, a user tends to seek a differently worded version of the same thing. So, this sort of “cognitive learning” may be treated differently than physical learning. Users tend to quickly tune out when they detect the same thing being repeated.

Visual reps include aspects of each of these learning areas and may be uniquely exploited to avoid the downfalls of either the pure physical or cognitive model reps. Visual reps do not involve moving the body so they differ from physical reps in that way. And visual reps do not include acquiring a fact-based or narrative account of a phenomenon like instructional videos do for certain movement skill techniques. Visual reps benefit from repetition of identical content (or at least very similar content pertaining to the same underlying phenomenon). In order to train movement skill, efforts may be made to ensure users stay bought into that repetition when they might be starting to feel that they should move on as they would for other cognitive learning.

In a visual reps course, it may be useful for the user to view some of the lessons multiple times (in an example, three of the lessons must be viewed nine times each) where these multiple viewings may be clustered together. In another example, in an analogous effort to get a user to deeply ingrain some specific content related to a certain subset of the set of subskills that comprise a skill, a user may view many somewhat different lessons one time each where all of them pertain to the same subskills (e.g., all teach the same “biomechanical content”). In the case where the same content is repeated, this repetition within the process of working through a course may be a unique feature of visual reps courses. Just as physical reps are used in current methods of physical skills training to repeatedly signal the body that it should reinforce internal components related to producing nervous system signals involved in creating a motion, visual reps may be intended to reinforce those same or similar components of the motor control system and thus may require many repetitions to have an appreciable effect.

This aspect of visual reps courses may be designed to adapt to the user's behavior while they work through the course in order to both keep them engaged and take advantage of an ordered learning scheme (e.g., characterized by later lessons building on the information provided in earlier lessons).

Content may be ordered in a scheme to direct the user through the critical to-be-learned information that the course is designed to provide. As discussed above, some content may be repeated. This repetition of content may be packaged into “lessons” where the lessons themselves are repeated. It may be packaged in some other scheme where portions of the video content in question, regardless of whether these portions are separated out as self-contained lessons, are repeated. When the repeated content is not separated out into lessons, segments may be repeated. Thus “lessons” or “segments” may be repeated (or a combination of lessons or segments may be repeated). For readability, the below description includes the “lessons” repetition, but may be applied to segments or both.

In order to ensure users first know certain ideas that themselves help them to make sense of later ideas, an intended order may be used. This intended order may be embodied in the form of a list of lessons where the first lesson that should be viewed is assigned the number one, the next lesson gets the number two, and so on, such that the earlier on a lesson is to be viewed the lower number. This scheme is the “Basic Order”. The Basic Order is not concerned with repetition of individual lessons. In other words, the Basic Order orders the lessons to reflect a “prerequisite structure” and includes each lesson only once.

On the other hand, when some or all lessons are to be repeated, an order of lessons that includes lessons multiple times may be established. This ordering may be embodied in the form of a “Template Order”. The first viewing of any lesson in the Template Order may respect the Basic Order. This means that, within the Template Order, when viewing, for example, the sixth lesson in the Basic Order for the first time, the user will have viewed each of lessons one through five in the Basic Order at least once. Aside from this requirement to respect the Basic Order, the Template Order may be arbitrarily mixed-up in terms of the order of lessons presented (in-particular, when presenting lessons for the second, third, or more, time).

This Template Order may be implemented within a system that allows the user to deviate or “skip ahead”, in an example. In this example, a lesson that is “recommended” to the user view may be skipped, and reverted to in subsequent recommendations. Further, as is described below, the system may itself adjust from the Template Order in what is recommended when triggered by tracking of user behavior. This may be based on the user behavior meeting certain conditions. The Template Order may resume when those conditions no longer hold. In the scheme of a visual reps course, when the intent is for the user to watch a certain lesson a certain number of times, the Template Order may account for this by including that lesson that number of times within its ordered sequence of lessons.

The Template Order may direct the user through a course by presenting some number of lessons to complete a course (e.g., ten) to the user. Some of these may give background information about the course, such as instructions for how to best use the course, neuroscience that makes the course effective, reasons why the technique is useful one, or the like. Other lessons may be “visual reps lessons” that may allow the user to observe rich visual and auditory information about key aspects of the technique and to participate in generating mental imagery related to those same aspects.

This set of lessons may be placed in a Basic Order and the Basic Order along with a “Repetition Guide” that defines how many times each lesson should be repeated in order to complete the course, may be used to establish a Template Order for the course.

The system may have a default expected cadence built into it. This cadence may include a number of lesson viewings per day or week. In an example the number is one lesson per day.

The system may calculate running averages of lessons watched per day over different numbers of days. In an example, after three days, the system may calculate a 3-day running average and may continue to maintain this metric. In an example, after seven days, it may calculate a 7-day running average and continue to maintain this metric. When the 3-day and the 7-day averages are close, the system may stay with the Template Order. When the 3-day average is well below the 7-day, it may adjust to preferentially choose the lesson that is earlier in the Basic Order of the lessons. It may do this in order to refresh the user on this more foundational, pre-requisite content since the user has been less “in-touch” with course material in recent days. This is to say that if the 3-day and 7-day averages were not divergent, the system may have stuck with the next step in the Template Order where that step included moving on to a next lesson in the Basic Order but that the slowing cadence of the user means the user may benefit from refreshing on the most recent lesson or a previous lesson (in the Basic Order) before moving on in the Template Order.

In another example, when the 3-day is above or well above the 7-day average, the sytem may adjust to avoid playing the same lesson two times in three viewings when it otherwise would have according to the Template Order. Independent of the 3-day and 7-day averages, repeating the same lesson may be avoided in a 24-hour period unless and until the user has viewed so many lessons in that period that the end of the Basic Order is achieved. When the Basic Order is completed, the progression may include returning to the most “prerequisite” lesson in the course which still has required watches before the user completes the course.

In an example, the system may compare the 7-day running average to the expected cadence. In an example, the expected cadence may be one lesson per day. Similar to the 3-day to 7-day comparison above, if the 7-day running average is significantly below the expected cadence, the system may not advance in the Basic Order of lessons in the course even when it may have otherwise based on the Template Order and may instead play the most recently watched video again to refresh. Or if the 7-day running average is above the expected cadence, the system may be triggered to move the user to a lesson that is further along in the Basic Order than it would have otherwise based on the template.

In another example, logic may be used that mixes comparisons between the expected cadence, the 7-day running average, and the 3-day running average in order to advance the user through the course in a way that keeps their engagement but preserves the value of pre-requisite content being displayed and possibly displayed repeatedly prior to content that comes later and is designed to be at least partially dependent upon those pre-requisites.

In another example, the system may consider the rate of change of the 7-day average. If this average is increasing in recent days that indicates an uptick in lesson viewing. The reverse is true for a decrease in the 7-day average. These may indicate a need to repeat content more or to move through the Basic Order a bit faster just as the other triggers described above may.

All of this may be subject to the constraints of the total number of viewings for any given lesson to complete the course (in other words, to respect those constraints, the system may not select a lesson that has already been viewed the total required number of times needed to complete the course and may otherwise default to the “position” in the Template Order that the system had been in prior to the triggered adjustment). Once the required viewings for each lesson in a course has been met, the system that guides the user through the lessons may persist. Beyond the point where the user has met all of the lesson-viewing-requirements, the system may retain the same logic about progressing through the visual reps lessons based on user behavior and the Template Order, except that it may ignore any limitations related to the lesson-viewing-requirements.

These schemes may use the Template Order and user behavior to select a lesson for the user to watch. There are several ways to leverage this selection of a lesson to the user's benefit. One of those is a forced-access system, meaning that it may only let the user watch the lesson chosen. Another is a recommendation system where a next lesson is indicated to the user, but the user is allowed a choice to deviate from the recommended selection. Another is a cue-up system where the lesson the system selects is cued up to play at the touch of a button, and the user may opt out of the cued-up lesson and into another lesson by doing some extra work. Another alternative includes a reminder system where the user receives a notification that reminds the user to view a lesson in the next few hours and indicates which lesson the user is recommended to view next.

Aside from any logical conflicts between the forced access system and the recommendation system or the cue-up system, elements of these can be implemented together, resulting in blended systems.

Note that the user may be able to see a dashboard with visualizations of their progress through the course displayed to them through some interface provided in an application that may or may not be the same application which delivers course content to them. They may see their 3-day and 7-day averages of completed lessons, viewing time, time spent doing physical reps over a recent time period (this may be self reported), other metrics, and their progress in meeting the lesson viewing requirements of the course.

Another scheme for deciding when the user has met the requirements of a course is to test for learning. The system may present the user with test material related to the critical information provided within certain lessons and when they meet a certain standard in answering questions in the test they may be deemed to have learned enough to move on. This may be combined with a Template Order which may incorporate the tests right into the order and may not allow access to later portions of the course until a test is passed. Those later portions of the course may include lessons that the user already passed the tests on as refreshers or to reinforce the content.

In an example, the visual reps lessons may include meditative sections which guide the user through a series of exercises which constitute directing the user to focus on certain portions of their body, actions the user may take with their body including clenching muscles, or other mental tasks as a way of enhancing the level of concentration and focus prior to viewing the lesson. This segment may be used prior to each visual reps lesson, but the system may give the user an option to opt out of that segment if they are time constrained and need to get their visual reps lesson done that day without using this segment.

FIG. 6 illustrates a block diagram of an example machine 600 upon which any one or more of the processes discussed herein may perform in accordance with some embodiments. In alternative embodiments, the machine 600 may operate as a standalone device or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Machine (e.g., computer system) 600 may include a hardware processor 602 (e.g., a central processing unit (CPU), a graphics processing unit (GPU), a hardware processor core, or any combination thereof), a main memory 604 and a static memory 606, some or all of which may communicate with each other via an interlink (e.g., bus) 608. The machine 600 may further include a display unit 610, an alphanumeric input device 612 (e.g., a keyboard), and a user interface (UI) navigation device 614 (e.g., a mouse). In an example, the display unit 610, input device 612 and UI navigation device 614 may be a touch screen display. The machine 600 may additionally include a storage device (e.g., drive unit) 616, a signal generation device 618 (e.g., a speaker), a network interface device 620, and one or more sensors 621, such as a global positioning system (GPS) sensor, compass, accelerometer, or other sensor. The machine 600 may include an output controller 628, such as a serial (e.g., Universal Serial Bus (USB), parallel, or other wired or wireless (e.g., infrared (IR), near field communication (NFC), etc.) connection to communicate or control one or more peripheral devices (e.g., a printer, card reader, etc.).

The storage device 616 may include a machine readable medium 622 on which is stored one or more sets of data structures or instructions 624 (e.g., software) embodying or utilized by any one or more of the processes or functions described herein. The instructions 624 may also reside, completely or at least partially, within the main memory 604, within static memory 606, or within the hardware processor 602 during execution thereof by the machine 600. In an example, one or any combination of the hardware processor 602, the main memory 604, the static memory 606, or the storage device 616 may constitute machine readable media.

While the machine readable medium 622 is illustrated as a single medium, the term “machine readable medium” may include a single medium or multiple media (e.g., a centralized or distributed database, or associated caches and servers) configured to store the one or more instructions 624. The term “machine readable medium” may include any medium that is capable of storing, encoding, or carrying instructions for execution by the machine 600 and that cause the machine 600 to perform any one or more of the processes of the present disclosure, or that is capable of storing, encoding or camying data structures used by or associated with such instructions. Non-limiting machine readable medium examples may include solid-state memories, and optical and magnetic media.

The instructions 624 may further be transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., institute of Electrical and Electronics Engineers (IEEE) 802.11 family of standards known as Wi-Fi@, IEEE 802.16 family of standards known as WiMax@), IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MIMO), or multiple-input single-output (MISO) processes. The term “transmission medium” shall be taken to include any intangible medium that is capable of storing, encoding or carrying instructions for execution by the machine 600, and includes digital or analog communications signals or other intangible medium to facilitate communication of such software.

Example 1 is a system comprising: processing circuitry; memory, communicatively coupled to the processing circuitry, the memory including instructions, which when executed by the processing circuitry, cause the processing circuitry to: retrieve a skill training data file selected by a user, the skill training data file including image frames and sounds related to a skill; and output the skill training data file for display on a user interface; and a display device to display the skill training data file, the skill training data file including a set of video portions displayed in a sequence including a first passive video portion, and a second video portion including visual or audible instructions directing a viewer to imagine body sensations corresponding to images shown in the second video portion, the body sensations representative of muscle movements used to perform the skill.

In Example 2, the subject matter of Example 1 includes, wherein the skill training data file includes an iteration of the first passive video portion, and the second video portion.

In Example 3, the subject matter of Examples 1-2 includes, wherein the sounds include music, and wherein the music is played during the second video portion.

In Example 4, the subject matter of Examples 1-3 includes, wherein the second video portion includes the image frames and sound of the first passive video portion.

In Example 5, the subject matter of Example 4 includes, wherein the visual or audible instructions are displayed or played before playing the image frames and sound.

In Example 6, the subject matter of Examples 1-5 includes, wherein the body sensations include a weight transfer body sensation, a muscle strain body sensation, or a spatial position body sensation.

In Example 7, the subject matter of Examples 1-6 includes, wherein the skill training data file includes a third video portion, the third video portion including instructions to the viewer to imagine the first video portion without showing the first video portion.

In Example 8, the subject matter of Example 7 includes, wherein to display the skill training data file, the display device is to display the first passive video portion, the third video portion, and the second video portion in that order.

Example 9 is a system comprising: processing circuitry; memory, communicatively coupled to the processing circuitry, the memory including instructions, which when executed by the processing circuitry, cause the processing circuitry to: retrieve a skill training data file selected by a user, the skill training data file including image frames and sounds related to a skill; and output the skill training data file for display on a user interface; and a display device to display the skill training data file, the skill training data file including a set of video portions displayed in a sequence including: a first passive video portion including a set of images, a second video portion including visual or audible instructions directing a viewer to imagine body sensations corresponding to the set of images repeated in the second video portion, the body sensations representative of muscle movements used to perform the skill, and a third video portion, the third video portion including visual or audible instructions to the viewer to imagine the skill depicted in the set of images from the first video portion without showing the set of images from the first video portion.

In Example 10, the subject matter of Example 9 includes, wherein the display device is configured to display the first passive video portion, then the third video portion, and then the second video portion in that order.

In Example 11, the subject matter of Examples 9-10 includes, wherein the display device is configured to display the first passive video portion, then the second video portion, and then the third video portion in that order.

In Example 12, the subject matter of Examples 9-11 includes, wherein the visual or audible instructions in the second video portion are displayed or played before playing the image frames and sound.

In Example 13, the subject matter of Examples 9-12 includes, wherein the visual or audible instructions in the third video portion are played while displaying a blank screen.

In Example 14, the subject matter of Examples 9-13 includes, wherein the body sensations include a weight transfer body sensation, a muscle strain body sensation, or a spatial position body sensation.

Example 15 is a system comprising: processing circuitry; memory, communicatively coupled to the processing circuitry, the memory including instructions, which when executed by the processing circuitry, cause the processing circuitry to: retrieve a skill training data file selected by a user, the skill training data file including image frames and sounds related to a skill; and output the skill training data file for display on a user interface; and a display device to display the skill training data file, the skill training data file including a set of video portions displayed in a sequence including a first passive video portion, and a second video portion including visual or audible instructions directing a viewer to imagine body sensations corresponding to images shown in the second video portion including at least one of a weight transfer body sensation, a muscle strain body sensation, or a spatial position body sensation, the body sensations representative of muscle movements used to perform the skill.

In Example 16, the subject matter of Example 15 includes, wherein the weight transfer body sensation, the muscle strain body sensation, or the spatial position body sensation is selected for inclusion in the skill training data file based on the skill.

In Example 17, the subject matter of Examples 15-16 includes, wherein the skill training data file is stored in a database of skill training data files, the database including at least one skill training data file corresponding to each of the weight transfer body sensation, the muscle strain body sensation, and the spatial position body sensation.

In Example 18, the subject matter of Examples 15-17 includes, wherein the second video portion includes the image frames and sound of the first passive video portion.

In Example 19, the subject matter of Examples 15-18 includes, wherein the skill training data file includes a third video portion, the third video portion including instructions to the viewer to imagine the first video portion without showing the first video portion.

In Example 20, the subject matter of Example 19 includes, wherein to display the skill training data file, the display device is to display the first passive video portion, the third video portion, and the second video portion in that order.

Example 21 is at least one machine-readable medium including instructions that, when executed by processing circuitry, cause the processing circuitry to perform operations to implement of any of Examples 1-20.

Example 22 is an apparatus comprising means to implement of any of Examples 1-20.

Example 23 is a system to implement of any of Examples 1-20.

Example 24 is a method to implement of any of Examples 1-20.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

What is claimed is:

1. A system comprising:
processing circuitry;
memory, communicatively coupled to the processing circuitry, the memory including instructions, which when executed by the processing circuitry, cause the processing circuitry to:
retrieve a skill training data file selected by a user, the skill training data file including image frames and sounds related to a skill; and
output the skill training data file for display on a user interface; and
a display device to display the skill training data file, the skill training data file including a set of video portions displayed in a sequence including a first passive video portion, and a second video portion including visual or audible instructions directing a viewer to imagine body sensations corresponding to images shown in the second video portion, the body sensations representative of muscle movements used to perform the skill.

2. The system of claim 1, wherein the skill training data file includes an iteration of the first passive video portion, and the second video portion.

3. The system of claim 1, wherein the sounds include music, and wherein the music is played during the second video portion.

4. The system of claim 1, wherein the second video portion includes the image frames and sound of the first passive video portion.

5. The system of claim 4, wherein the visual or audible instructions are displayed or played before playing the image frames and sound.

6. The system of claim 1, wherein the body sensations include a weight transfer body sensation, a muscle strain body sensation, or a spatial position body sensation.

7. The system of claim 1, wherein the skill training data file includes a third video portion, the third video portion including instructions to the viewer to imagine the first video portion without showing the first video portion.

8. The system of claim 7, wherein to display the skill training data file, the display device is to display the first passive video portion, the third video portion, and the second video portion in that order.

9. A system comprising:
processing circuitry;
memory, communicatively coupled to the processing circuitry, the memory including instructions, which when executed by the processing circuitry, cause the processing circuitry to:
retrieve a skill training data file selected by a user, the skill training data file including image frames and sounds related to a skill; and
output the skill training data file for display on a user interface; and
a display device to display the skill training data file, the skill training data file including a set of video portions displayed in a sequence including:
a first passive video portion including a set of images,
a second video portion including visual or audible instructions directing a viewer to imagine body sensations corresponding to the set of images repeated in the second video portion, the body sensations representative of muscle movements used to perform the skill, and
a third video portion, the third video portion including visual or audible instructions to the viewer to imagine the skill depicted in the set of images from the first video portion without showing the set of images from the first video portion.

10. The system of claim 9, wherein the display device is configured to display the first passive video portion, then the third video portion, and then the second video portion in that order.

11. The system of claim 9, wherein the display device is configured to display the first passive video portion, then the second video portion, and then the third video portion in that order.

12. The system of claim 9, wherein the visual or audible instructions in the second video portion are displayed or played before playing the image frames and sound.

13. The system of claim 9, wherein the visual or audible instructions in the third video portion are played while displaying a blank screen.

14. The system of claim 9, wherein the body sensations include a weight transfer body sensation, a muscle strain body sensation, or a spatial position body sensation.

15. A system comprising:
processing circuitry;
memory, communicatively coupled to the processing circuitry, the memory including instructions, which when executed by the processing circuitry, cause the processing circuitry to:
retrieve a skill training data file selected by a user, the skill training data file including image frames and sounds related to a skill; and
output the skill training data file for display on a user interface; and
a display device to display the skill training data file, the skill training data file including a set of video portions displayed in a sequence including a first passive video portion, and a second video portion including visual or audible instructions directing a viewer to imagine body sensations corresponding to images shown in the second video portion including at least one of a weight transfer body sensation, a muscle strain body sensation, or a spatial position body sensation, the body sensations representative of muscle movements used to perform the skill.

16. The system of claim 15, wherein the weight transfer body sensation, the muscle strain body sensation, or the spatial position body sensation is selected for inclusion in the skill training data file based on the skill.

17. The system of claim 15, wherein the skill training data file is stored in a database of skill training data files, the database including at least one skill training data file corresponding to each of the weight transfer body sensation, the muscle strain body sensation, and the spatial position body sensation.

18. The system of claim 15, wherein the second video portion includes the image frames and sound of the first passive video portion.

19. The system of claim 15, wherein the skill training data file includes a third video portion, the third video portion including instructions to the viewer to imagine the first video portion without showing the first video portion.

20. The system of claim 19, wherein to display the skill training data file, the display device is to display the first passive video portion, the third video portion, and the second video portion in that order.
